(19) United States
(12) Patent Application Publication
(10) Pub. No.: US 2007/0059796 A1
(43) Pub. Date: Mar. 15, 2007

(54) Binding motif and methods of regulating cell function

(75) Inventors: Mark Guthridge, Brompton (AU); Hayley Ramshaw, Adelaide (AU); Frank Stomski, Redwood Park (AU); Angel Lopez, Medindie (AU)

Correspondence Address: DARBY & DARBY P.C., P. O. BOX 5257, NEW YORK, NY, 10150-5257, US

(73) Assignee: Medvet Science Pty. Ltd., Adelaide (AU)

(21) Appl. No.: 10/595,559

(22) PCT Filed: Oct. 27, 2004
(86) PCT No.: PCT/AU04/01480

§ 371 Date: Sep. 11, 2006

(30) Foreign Application Priority Data
Oct. 27, 2003 (AU) 2003905932

(51) Int. Cl. A61K 38/17; C07K 14/715; C07H 21/04; C12P 21/06

(52) U.S. Cl. 435/69.1; 435/320.1; 435/325; 530/350; 536/23.5; 514/12

(57) ABSTRACT

The present invention relates to a binding motif and methods of regulating cell function which methods target a single amino acid residue preferably a Tyr in a binding motif equivalent to a motif of the common beta chain (βc) of the GM-CSF/IL-3/IL-5 receptor. Preferably, the cell functions affect cell survival and proliferation in cells. The methods can be used for treatments of conditions relating to cell survival and proliferation and can be used to expand progenitor cells, for instance, for transplantation purposes.

### FIELD OF THE INVENTION

The present invention relates to a binding motif and methods of regulating cell function which methods target a single amino acid residue in a binding motif equivalent to a motif of the common beta chain (βc) of the GM-CSF/IL-3/IL-5 receptor. Preferably, the cell functions affect cell survival and proliferation in cells. The methods can be used for treatments of conditions relating to cell survival and proliferation and can be used to expand progenitor cells, for instance, for transplantation purposes.

### BACKGROUND

The number of haematopoietic cells generated in vitro and in vivo is tightly regulated by the integration of survival, proliferation and differentiation signals that emanate from growth factor receptors. Defining the molecular mechanisms regulating these processes is critical for the design of new strategies to expand haematopoietic progenitor cells and their progeny for bone marrow transplantation, and for our understanding of leukaemia, myeloproliferative diseases and chronic inflammation where the normal balance of cell production and function has broken down.

Although many cytokines such as IL-3, GM-CSF and IL-5 and growth factors such as PDGF and IGF-1 were initially discovered as mitogens by virtue of their ability to promote cell proliferation, many of these factors were later also found to be potent regulators of cell survival through their ability to suppress programmed cell death or apoptosis. These biological activities are regulated by the binding of the cytokine or growth factor to its cognate cell surface receptor which initiates an ordered series of signalling events that includes receptor dimerization, the activation of tyrosine kinases followed by the tyrosine phosphorylation of the receptor cytoplasmic tail, the binding of multiprotein signalling complexes to receptor phosphotyrosine residues via src-homology 2 (SH2) domains or phosphotyrosine-binding (PTB) domains and the activation of downstream signalling cascades that promote a cellular response.

Many factors including cytokines can contribute to cell survival and proliferation and the regulation of these to easily manipulate control is not a simple matter. Identification of one controlling factor can assist in the development of useful treatments and diagnosis of conditions where cell production and function are out of balance.

The action of signalling molecules such as cytokines has been poorly understood. It is apparent that these cellular proteins can switch on activities within cells. However, the actual triggering mechanisms and how these are relayed to culminate in their final activities is not known. Cell cycles are clearly involved but the link between the signalling molecule and receptor and actions such as cell survival, proliferation, and differentiation is unclear.

Proteins including human granulocyte-macrophage colony-stimulating factor (GM-CSF), interleukin-3 (IL-3), and IL-5 are capable of stimulating normal and transformed hematopoietic cells. With each, the initiating event for signal transduction is the binding of the protein to its surface receptors. These receptors may be composed of subunits such as the α chain and a common β chain (β_{c}). Engagement of β_{c} by the binding of the cytoplasmic protein to surface receptors results in the stimulation of cell survival, proliferation, and differentiation and mature cell effector function in the appropriate lineage, a fact that emphasises the major signalling role played by β_{c} in mediating receptor induced biological activities.

One of the first events in activation of receptors and in the initiation of the signalling cascade is tyrosine phosphorylation of β_{c}. This is a common theme among receptor signalling subunits and can be seen in homodimeric receptors such as the erythropoietin (EPO) receptor, thrombopoietin (TPO) receptor, and granulocyte colony-stimulating factor (G-CSF) receptor as well as in heterodimeric receptors such as in the IL-6 and IL-2 receptors, and in the GM-CSF, IL-3, and IL-5 receptor systems.

Tyrosine phosphorylation of receptor signalling subunits appeared to be a critical step in the creation of docking sites for the association of signalling molecules. Despite the perceived importance of tyrosine phosphorylation of receptors it is becoming apparent in some cells that signalling can proceed in its absence. This is demonstrated in the EPO and TPO receptors, in which the substitution of all tyrosines failed to abolish their activities.

It has been unclear until now how the binding of proteins to their receptors can result in the specialised functions associated with these molecules and their receptors.

### SUMMARY OF THE INVENTION

In a first aspect of the present invention, there is provided a binding motif capable of binding to a cytoplasmic protein said motif consisting of the following amino acid sequence:
N—X—X—Y
wherein X is any residue, and Y is tyrosine or an equivalent thereof.

A single amino acid corresponding to Tyr577 of βc of the GM-CSF receptor has been identified as a controlling factor in the regulation of cellular activities. For this reason, this invention targets this amino acid residue for modulating cellular activity associated with the GM-CSF receptor of the GM-CSF cytokine.

In a preferred embodiment, there is provided a binding motif of a receptor. The receptor may be any receptor that is capable of binding to an extracellular molecule or protein and which mediates its function through the binding of a cytoplasmic molecule or protein such as Shc, or any cytoplasmic molecule or protein capable of binding a further signalling molecule which activates a cascade of events leading to cell signalling pathways and biological functions such as mitogenesis, proliferation, transformation, differentiation and cell survival, or any other cytoplasmic molecule or protein.

In another aspect of the present invention there is provided a method of modulating activity in a cell, said method including:
introducing a modification to a binding motif capable of binding to a cytoplasmic protein said motif consisting of the following sequence:
   N—X—X—Y
   wherein X is any residue, and Y is tyrosine.

In another aspect of the present invention there is provided a method modulating activity in a cell, said method including:
introducing a modification to a tyrosine of a binding motif capable of binding to a cytoplasmic protein said motif consisting of the following sequence:
   N—X—X—Y
   wherein X is any residue, and Y is tyrosine and wherein the tyrosine is equivalent to Tyr577 of the common beta chain (βc) of the GM-CSF receptor or an equivalent thereof.

Accordingly, in yet another preferred embodiment, the invention provides a method of modulating activity in a cell, said method including
modifying phosphorylation of a tyrosine of a binding motif capable of binding to a cytoplasmic protein said motif consisting of the following sequence:
   N—X—X—Y
   wherein X is any residue, and Y is tyrosine.

In yet another preferred aspect of the present invention, there is provided a method of increasing cell growth, said method including
inhibiting activation of a tyrosine of a binding motif capable of binding to a cytoplasmic protein said motif consisting of the following sequence:
   N—X—X—Y
   wherein X is any residue, and Y is tyrosine.

In yet another aspect of the present invention there is provided a method of transplantation of cells or enhancing transplantation efficiency, said method including
inhibiting activation in one or more cells of a tyrosine of a binding motif capable of binding to a cytoplasmic protein said motif consisting of the following sequence:
   N—X—X—Y
   wherein X is any residue, and Y is tyrosine; and
transplanting the cells into a patient in need of such treatment.

In another aspect of the present invention there is provided a method of improving wound healing in a patient, said method including
inhibiting activation of a tyrosine of a binding motif capable of binding to a cytoplasmic protein said motif consisting of the following sequence:
   N—X—X—Y
   wherein X is any residue, and Y is tyrosine in a region of the wound.

In yet another aspect of the present invention, there is provided a method for screening cell growth promoting compounds, said method including
providing a cell in which phosphorylation of the Tyr577 or an equivalent has been induced;
exposing the cell to the compound; and
assessing colony formation of the cells.

### FIGURES

FIG. 1 shows Y577F mutation leads to enhanced numbers of colonies in response to GM-CSF.

FIG. 2 shows Y577F point mutation can give rise to larger colonies than wild type GM-CSF receptor beta chain.

FIG. 3 shows foetal liver cells transduced with Y577F mutant of the beta chain form greater numbers of colonies at all concentrations of GM-CSF.

FIG. 4a shows a Delta Assay of cells cultured for 7 days prior to colony formation.

FIG. 4b shows a Delta Assay of cells cultured for 14 days prior to colony formation.

FIG. 5 shows CTL cells expressing the human GM-CSF receptor have greater response to GM-CSF when Y577 is mutated to phenylalanine.

FIG. 6 shows the amino acid sequence of βc.

### DETAILED DESCRIPTION OF THE INVENTION

In a first aspect of the present invention, there is provided a binding motif capable of binding to a cytoplasmic protein said motif consisting of the following amino acid sequence:
N—X—X—Y
wherein X is any residue, and Y is tyrosine or an equivalent thereof.

The term “motif” as used herein, means a distinctive amino acid sequence which is conserved and forms a unit in which the amino acids interact.

Signalling molecules may be molecules involved in cellular pathways such as but not limited to those pathways involved in proliferation, survival or differentiation. Examples of such pathways may include the JAK/STAT pathway, the ras/MAP kinase pathway or the PI-3-Kinase pathway. All pathways may be involved directly or indirectly with these functions.

The term “cell signalling pathways” as used herein includes all cellular pathways and cellular reactions which contribute to the functioning of the cell. It is not restricted to reactions that arise from cytokine mediated binding to the receptor. However, it is most preferred that the activities are activated by cytokine binding.

The cytoplasmic protein will be one that interacts with a cell receptor through a particular amino acid, namely tyrosine, however, it is preferred that the cytoplasmic proteins that bind to the amino acid are selected from the group including 14-3-3 protein, Shc, SHIP-2, WW-domain of the prolyl isomerase, Pin1 and the ubiquitin ligase, NEDD4 and any cytoplasmic protein capable of binding a further signalling molecule which activates a cascade of events preferably leading to cell signalling pathways or other pathways and biological functions in a cell such as mytogenesis, proliferation, transformation, differentiation and cell survival. More preferably, the cytoplasmic protein is Shc. Most preferably, Shc will bind to tyrosine.

The term “signalling molecule” is any molecule that can signal an activation in the signalling pathway.

Shc will bind to Tyr via its PTB domain and has the potential to both positively and negatively regulate intracellular signalling. For example, in addition to its suggested positive role in promoting signalling via the Ras/Map kinase pathway through the recruitment of grb2/sos and via the PI 3-kinase pathway through the recruitment of a grb2/GAB2/PI 3-kinase complex, Shc is also known to recruit negative regulators of signalling including the phosphatases SHP2 and SHIP.

The cytoplasmic proteins which bind to the amino acid will in turn bind to further signalling molecules which can activate a cascade of events leading to cell signalling pathways and biological functions such as, but not limited to, mitogenesis, proliferation, transformation, differentiation and cell survival or any other cytoplasmic molecule or protein.

Preferably, the tyrosine residue can react with cytoplasmic proteins and in turn the tyrosine and cytoplasmic protein can interact to activate cellular activity.

More preferably, the cytoplasmic protein that binds to tyrosine is Shc, or SHIP-2.

In a preferred embodiment, there is provided a binding motif of a receptor. The receptor may be any receptor that is capable of binding to an extracellular molecule or protein and which mediates its function through the binding of a cytoplasmic molecule or protein such as Shc, or any cytoplasmic molecule or protein capable of binding a further signalling molecule which activates a cascade of events leading to cell signalling pathways and biological functions such as mitogenesis, proliferation, transformation, differentiation and cell survival, or any other cytoplasmic molecule or protein.

A receptor as used herein may be selected from the group including:
(1) GM-CSF/IL-3/IL-5 receptor
(2) IL6 human interleukin-6 receptor beta chain precursor (IL-6R-beta)
(3) LEPR human leptin receptor precursor (LEP-R) (OB RECEPTOR) (OB-R).
(4) TNR2 human tumor necrosis factor receptor 2 precursor (tumor necrosis factor
(5) VGR1 human vascular endothelial growth factor receptor 1 precursor
(6) TRK3 human receptor protein-tyrosine kinase TKT precursor (EC 2.7.1.112)
(7) Q01974 protein-tyrosine kinase transmembrane receptor ROR2 precursor
(8) FGR1 human basic fibroblast growth factor receptor 1 precursor (BFGF-R)
(9) Q15426 protein-tyrosine phosphatase, receptor-type, H precursor (EC 3.1.3.48)
(10) PTPM human protein-tyrosine phosphatase mu precursor (EC 3.1.3.48) (R-PTP-MU).
(11) PDGS human alpha platelet-derived growth factor receptor precursor (EC 2.7.1.112)
(12) FGR4 human fibroblast growth factor receptor 4 precursor (FGFR4) (EC 2.7.1.112)
(13) FGR2 human fibroblast growth factor receptor 2 precursor (FGFR-2) (EC 2.7.1.112)
(14) Q13635 patched protein homolog (PTC)
(15) MANR human macrophage mannose receptor precursor.
(16) LRP2 human low-density lipoprotein receptor-related protein 2 precursor (megalin)
(17) IDD human integral membrane protein dgcr2/idd precursor (KIAA0163)
(18) AMFR human autocrine motility factor receptor precursor (AMF receptor) (gp78)
(19) ACH5 human neuronal acetylcholine receptor protein, alpha-5 chain precursor.
(20) KKIT human: stem cell growth factor receptor (proto-oncogene tyrosine-protein kinase kit) (C-KIT) (CD117 antigen)
(21) TPOR human: thrombopoietin receptor precusor (TPO—R) (myeloproliferative leukemia protein (C-MPL). TPOR or MPL.
(22) TPOR mouse: thrombopoietin receptor precursor (TPO—R) (myeloproliferative leukemia protein) (C-MPL). TPOR or MPL.
(23) Acetylcholine R
(24) Acetylcholine R alpha-5
(25) C—C chemokine receptor 6
(26) Middle T antigen
(27) integrin alpha 1
(28) FGFR2 (KGF R)
(29) FGFR1 (fig)
(30) FGFR5
(31) Erb4
(32) Vaccinia virus protein A36R
(33) Macrophage mannose R (MRC1)
(34) LDLR
(35) VLDL (rat)
(36) LRP1 low density lipoprotein receptor-related protein 1
(37) integrin beta 1
(38) integrin beta 7
(39) integrin beta 3
(40) integrin beta 5
(41) integrin beta 6
(42) G-CSFR1 (second)
(43) G-CSFR1
(44) g-csf-r
(45) IL-6B (gp130)
(46) LeptinR
(47) ProlactinR
(48) insulinR
(49) irs-1
(50) IGFI R
(51) flt3 R
(52) VEGFR2 (FLK1)
(53) PDGF R-alpha
(54) IL-9R
(55) Beta R
   or a functional equivalent or analogue thereof.

The receptor is preferably a cytokine receptor. More preferably it is the GM-CSF/IL-3/IL-5 receptor which includes βc.

The binding capacity of the motif may be analysed by any binding studies or experiments available to the skilled addressee. Such experiments may include measuring the binding ability of a designated cytoplasmic protein to the binding motif. For instance electrophoretic mobility shift assays (EMSA or band shift assays) or foot print assays or pull down experiments are available to measure specific binding.

The motif (N—X—X—Y) can be identified by the presence of a tyrosine preferably in an amino acid sequence N—X—X—Y, and the ability to bind a designated cytoplasmic protein. The designated cytoplasmic protein may be Shc, SHIP-2 or any cytoplasmic protein capable of binding a further signalling molecule which activates a cascade of events leading to cell signalling pathways and biological functions such as mitogenesis, proliferation, transformation, differentiation and cell survival. More preferably, the cytoplasmic protein is Shc or SHIP-2.

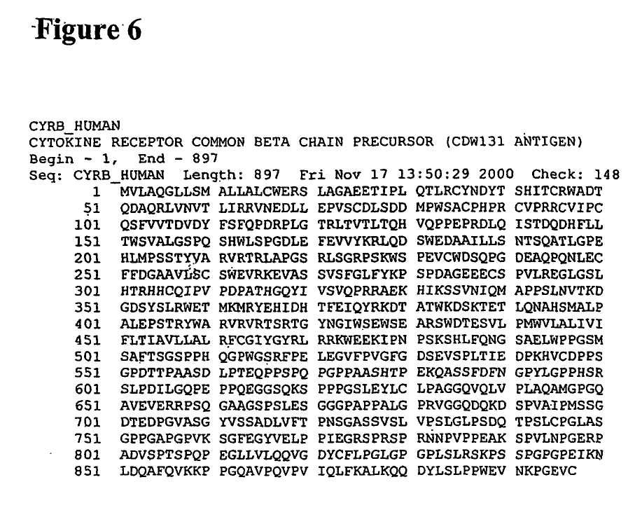

Preferably, the receptor is the GM-CSF/IL-3/IL-5 receptor which includes the common beta chain (β_{c}). It is found that the cytokines GM-CSF, IL-3 and IL-5 signal their actions through the surface receptor via the β_{c}. Most preferably, the binding motif comprises a sequence which includes the amino acids Tyr corresponding to amino acid Tyr577 of β_{c} according to FIG. 6 or a functional equivalent or analogue thereof.

The term “functional equivalent or analogue thereof” as used herein means a sequence which functions in a similar way but may have deletions, additions or substitutions that do not substantially change the activity or function of the sequence.

The common β chain (β_{c}) is a component of a cytokine receptor and is part of a signalling subunit of the receptor. It is thought that the cytokine signals its functions through β_{c}, initiating events which cascade and culminate in an identifiable biological function such as cell survival, proliferation, differentiation and mature cell effector functions. However, the present invention is not limited to motifs of β_{c} but includes motifs of receptors and other proteins within the cell having similar sequences to the β_{c} and including a tyrosine residue at an equivalent position. It is within the scope of this invention that the bidentate motif will have the structure identified above and through this structure, the cytokine may exert its effects on the cell. Preferably, the binding motif is found in the region of the βc which includes Tyr577. Having this as a guide the binding motif of all proteins having a similar motif which corresponds to the region of βc including Tyr577 are within the scope of this invention.

The region or motif including amino acid Tyr577 of β_{c} or functional equivalent thereof may include residues which preferably interact with a cytoplasmic protein selected from the group including 14-3-3 protein, Shc, SHIP-2, WW-domain of the prolyl isomerase, Pin1, and the ubiquitin ligase, NEDD4 or any cytoplasmic protein capable of binding a further signalling molecule which activates a cascade of events leading to cell signalling pathways and biological functions such as mitogenesis, proliferation, transformation, differentiation and cell survival. However the present invention is not limited to this sequence but includes other equivalent sequences capable of performing the same function.

Throughout the description and claims of this specification, use of the word “comprise” and variations of the word, such as “comprising” and “comprises”, is not intended to exclude other additives, components, integers or steps.

The bidentate motif may be present in any type of cell. The motif structure including the amino acid sequence as described above can be screened in any cell. However, preferably it is a cell that can be affected by GM-CSF or includes βc. Most preferably, the cell is one that is affected by binding of signalling molecules to βc which harbours Tyr, more preferably corresponding to Tyr577 of βc. Most preferably, the cell is a haematopoietic cell such as, but not limited to, lymphoid, myeloid and erythroid cells. Specifically, the lymphoid lineage, comprising B cells and T cells, produces antibodies, regulates cellular immunity, and detects foreign agents such as disease-causing organisms in the blood. The myeloid lineage, which includes monocytes, granulocytes, and megakaryocytes, monitors the blood for foreign bodies, protects against neoplastic cells, scavenges foreign materials, and produces platelets. The erythroid lineage includes red blood cells, which carry oxygen. Accordingly, because the bidentate motif most preferably affects the haematopoietic cell lines, it is within the scope of the present invention that cellular activities associated with any of these cell lines may also be modulated by targeting a modification to Tyr577 of βc of GM-CSF/IL-5/IL-3.

In another embodiment of the present invention, it is preferred that the motif is selected from any one of the following sequences:

NGPYLG...........PP..HSRSLP
NVHYRT...........P...KTHTMP
**RYFTQKEE.........TESGSGP
NKKYELQDRDVCE....P.RYRSVSEP
NPTYSVM..............RSHSYP
NIFYLIR...KSGSFPMPELKLSISFP
NEEYLDLSQ........PLEQYSPSYP
NQEYLDLSM........PLDQYSPSFP
NATYKVD...........VIQRTRSKP
NPEY................HSASSGP
NPDY.................WNHSLP
NPSYSSNPFVNYN....KTSICSKSNP
NTLY................FNSQSSP
NPVYQKTTEDEVHI...CHNQDGYSYP
NPVYLKTTEEDLSIDIG..RH.SASVG
NPTYKMYEGGEPDDVGGLLDADFALDPDKPTNFTNPVY
NPIY...............KSAVTTVV
NPLY...............KSAITTTV
NPLY...............KEATSTFT
NPLY...............RKPISTHT
NPLY...............RGSTSTFK
PGHYL...............RCDSTQP
VQTYVLQ..........GDPRAVSTQP
QVLYGQLL.............GSPTSP
HSGYRHQVPSVQVF.....SRSESTQP
WKMYEVYDA........KS.KSVSLP
KIPYFHA..........GGS.KCSTWP
ELDYCLKGLKL......P.S.RTWSPP
SGDYMPM............SPKSVSAP
SFYYSEENKLPEPEELDLEPENMESVP(LDPSASSSSLP)1283 =
survl.
EEIYIIM.....QSCWAFDSRKRPSFP
ISQYLQN............S.KRKSRP
GTAY................GLSRSQP
***YLPQEDWAP.........TSLTRP
LVAYIAFKRWNSCKQN...KQGANSRPVNQTPPPEGEKLHSDSGIS

The sequences may correspond to the following:

| | |
|---|---|
| betaR.... | NGPYLG...........PP..HSRSLP |
| Acetylcholine R (ISOFROM?) | NVHYRT...........P...KTHTMP |
| Acetylcholine R alpha-5 | **RYFTQKEE..........TESGSGP |
| C-C chemokine receptor 6 | NKKYELQDRDVCE....P.RYRSVSEP |
| Middle T antigen | NPTYSVM..............RSHSYP |
| Integrin alpha 1 | NIFYLIR...KSGSFPMPELKLSISFP |
| FGFR2 (KGF R) | NEEYLDLSQ........PLEQYSPSYP |
| FGFR1 (flg) | NQEYLDLSM........PLDQYSPSFP |
| FGFR5 | NATYKVD...........VIQRTRSKP |
| Erb4 | NPEY................HSASSGP |
| Erb4 (second) | NPDY.................WNHSLP |
| Vaccinia virus protein A36R | NPSYSSNPFVNYN....KTSICSKSNP |
| Macrophage mannose R (MRC1) | NTLY................FNSQSSP |
| LDLR | NPVYQKTTEDEVHI...CHNQDGYSYP |
| VLDL (rat) | NPVYLKTTEEDLSIDIG..RH.SASVG |
| LRP1 low density lipoprotein | NPTYKMYEGGEPDDVGGLLDADFALDPDKPTNFTNPVY |
| receptor-related protein 1 | |
| integrin beta 1 | NPIY...............KSAVTTVV (end of protein) |
| interin beta 7 | NPLY...............KSAITTTV (end of protein) |
| integrin beta 3 | NPLY...............KEATSTFT (end of protein) |
| integrin beta 5 | NPLY...............RKPISTHT (end of protein) |
| integrin beta 6 | NPLY...............RGSTSTFK |
| G-CSFR1 (second) | PGHYL...............RCDSTQP |
| G-CSFR1 | VQTYVLQ..........GDPRAVSTQP |
| g-csf-r | QVLYGQLL..........GSPTSP (CHECK?) |
| IL-6B (gp130) | HSGYRHQVPSVQVF.....SRSESTQP |
| leptinR. | WKMYEVYDA.........KS.KSVSLP |
| prolactinR... | KIPYFHA..........GGS.KCSTWP |
| insulinR | ELDYCLKGLKL......P.S.RTWSPP |
| irs-1.... | SGDYMPM............SPKSVSAP |
| IGFI R | SFYYSEENKLPEPEELDLEPENMESVP(LDPSASSSSLP) |
| flt3 R | EEIYIIM.....QSCWAFDSRKRPSFP |
| VEGFR2 (FLK1) | ISQYLQN............S.KRKSRP |
| PDGF R-alpha | GTAY................GLSRSQP |
| IL-9R | ***YLPQEDWAP.........TSLTRP |
| p75 NTR | LVAYIAFKRWNSCKQN...KQGANSRPVNQTPPPEGEKLHSDSGIS |

More particularly, the binding motif or an equivalent may have any one of the following sequences.

NGPY
NVHY
**RY
NKKY
NPTY
NIFY
NEEY
NQEY
NATY
NPEY
NPDY
NPSY
NTLY
NPVY
NPIY
NPLY
▪

The present invention has found that mutation of Tyr577 is required to abolish haematopoetic cell survival in response to GM-CSF. This would suggest that this residue is able to independently regulate cell survival.

Applicants have further found that Shc interacts with 14-3-3 via a Tyr 179 which is necessary for PI-3 kinase activity. Via this signalling molecule, further signalling pathways are activated leading to cellular activities such as mitogenesis, proliferation, transformation, differentiation, cell survival, chemotaxis, motility, enhanced phagocytosis, bacterial killing, superoxide production and cytotoxicity.

In another aspect of the present invention there is provided a method of modulating activity in a cell, said method including:
introducing a modification to a binding motif capable of binding to a cytoplasmic protein said motif consisting of the following sequence:
   N—X—X—Y
   wherein X is any residue, and Y is tyrosine.

In a preferred aspect of the present invention, there is provided a method of modulating activity in a cell, said method including
introducing a modification to a tyrosine of a binding motif capable of binding to a cytoplasmic protein said motif consisting of the following sequence:
   N—X—X—Y
   wherein X is any residue, and Y is tyrosine and wherein the tyrosine is equivalent to Tyr577 of the common beta chain (βc) of the GM-CSF receptor or an equivalent thereof.

The use of the term “equivalent to Tyr577 of the common beta chain (βc) of the GM-CSF receptor” or “Tyr 577 or an equivalent” means Tyr577 of βc or another residue, preferably tyrosine which is in a position that can be aligned with Tyr577 based on the amino acid sequence of the common βc as in FIG. 6. The equivalent is to behave in a similar manner and have the same effects as Tyr577 on the common βc.

A single amino acid corresponding to Tyr577 of βc of GM-CSF receptor has been identified as a controlling factor in the regulation of cellular activities. For this reason, this invention targets this amino acid residue and the motif surrounding and supporting this residue for modulating cellular activity.

The GM-CSF receptor is responsible for a number of cellular activities, most of which cascade from binding of cytoplasmic proteins to βc of the receptor. Engagement of the signalling molecules to βc results in the stimulation of a number of cellular activities.

The common β chain (β_{c}) is a component of a cytokine receptor and is a signalling subunit of the receptor. It is thought that the cytokine signals its functions through the β_{c}, initiating events which cascade and culminate in an identifiable biological function such as cell survival, proliferation, differentiation and mature cell effector functions. However, the present invention is not limited to β_{c} of GM-CSF but includes motifs of receptors having similar sequences to β_{c} and including a residue in a similar position to the Tyr577 residue.

“Cellular activity” or “activity in the cell” as used herein may be selected from the group including cell survival, proliferation, differentiation, mitogenesis, transformation, chemotaxis, motility, enhanced phagocytosis, enhanced bacterial killing, superoxide production and cytotoxicity. Preferably, the cellular activity is any activity directly related to the GM-CSF receptor. Most preferably, the cellular activity is cell survival and proliferation or activity that leads to cell growth and colony growth.

However, these activities can be modulated by targeting a Tyr577 or an equivalent residue of β_{c} as a controlling factor of the cellular activities. This residue has previously been implicated in coupling the receptor to the adaptor protein Shc and to the tyrosine phosphatase SHIP-2.

“Modulation” or “modulating” as used herein with respect to cellular activity means modifying, altering or changing the activity compared to unmodified levels. The activity may be increased or decreased. For instance, proliferation may be increased or decreased. The modulation may cause an enhancement or reduction of the cellular activity. In any case, the cellular activity is changed by virtue of modification of the receptor at amino acid residue Tyr577 of β_{c}.

Any cell may be affected by a modification to a tyrosine, preferably Tyr577 of the common beta chain (βc). However, it is generally a cell that can be affected by GM-CSF or includes βc. Most preferably, the cell is one that is affected by binding of signalling molecules to βc which harbours Tyr577. Most preferably, the cell is a haematopoietic cell such as, but not limited to, lymphoid, myeloid and erythroid cells. Specifically, the lymphoid lineage, comprising B cells and T cells, produces antibodies, regulates cellular immunity, and detects foreign agents such as disease-causing organisms in the blood. The myeloid lineage, which includes monocytes, granulocytes, and megakaryocytes, monitors the blood for foreign bodies, protects against neoplastic cells, scavenges foreign materials, and produces platelets. The erythroid lineage includes red blood cells, which carry oxygen. Because the Tyr577 most preferably affects the haematopoietic cell lines, it is within the scope of the present invention that cellular activities associated with any of these cell lines may also be modulated by targeting a modification to Tyr577 of βc of GM-CSF.

The cell as used herein may be an isolated cell or be a cell contained within tissue or bodily fluids. Preferably the cell is a haematopoetic cell as herein described. Generally the cell will have a receptor for a cytokine such as GM-CSF. Most preferably the cell will have the GM-CSF receptor and activate via βc.

The haematopoietic cells may be derived from any source, such as from the bone marrow or peripheral blood, cell cultures or tissue samples. They may be isolated by methods available to the skilled addressee.

The methods of the present invention require modulation of at least one cellular activity by introducing a modification to a Tyr, preferably an equivalent to Tyr577 of βc. Introduction of the modification as used in the present invention may include introducing a mutation to a residue equivalent to Tyr577 of βc or it may include transducing a βc mutation having a mutation at the position of Tyr577 on βc. Methods of blocking the effects of Tyr577 to modulate cellular activity are also within the scope of the present invention and in the spirit of introducing a modification to Tyr577. In this case it includes molecules that are directed to the Tyr577 residue to block binding of Shc or SHIP-2 to activate the cascade of events arising from the binding or it may include preventing phosphorylation of Tyr577.

Preferably Tyr577 is the sole target and modulation of the cellular activity is dependent on the modification of Tyr577 only.

In one preferred embodiment, the modification is by inducing a mutation at a position equivalent to Tyr577 on βc. The mutation may include a substitution, deletion, or insertion of another amino acid such that the position equivalent to Tyr577 on βc is debilitated and no longer functional in so far as it cannot perform its normal functions such as binding cytoplasmic proteins.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

“Deletions” result from the amino acid being physically removed. The position may be targeted by methods available to the skilled addressee as used in site directed mutagenesis.

“Insertions” may arise where a similar amino acid is not inserted but another amino acid is inserted. Hence it is a non-conservative amino acid change.

Preferably, the substitutions replace Tyr577 or an equivalent with another amino acid. Preferably the Tyr577 or an equivalent is replaced with phenylalanine (Tyr577Phe).

In yet another preferred embodiment, the modification of Tyr577 or an equivalent may be achieved using antagonists, inhibitors, mimetics or derivatives of the Tyr577 or an equivalent. The terms “antagonist” or “inhibitor”, as used herein, refer to a molecule which, when bound to Tyr577 or an equivalent, blocks or modulates the activity of Tyr577 or an equivalent. Antagonists and inhibitors may include proteins, nucleic acids, carbohydrates, antibodies or any other molecules including ligands which bind to Tyr577 or an equivalent. Other modulators of the activity and/or expression of Tyr577 or an equivalent include a range of rationally-designed, synthetic inhibitors.

Modifications at Tyr577 or an equivalent may be achieved by direct or indirect methods. Modulation of activity of Tyr577 or an equivalent may be achieved using direct methods known to those of skill in the art and include, but are not limited to, knockout technology, targeted mutation, gene therapy. Indirect methods for modulating activity of Tyr577 or an equivalent may include targeting upstream or downstream regulators such as regulators of the cytoplasmic protein Shc or SHIP-2.

In yet another preferred embodiment, the modification of the Tyr577 or an equivalent that modulates cellular activity is achieved by introducing a construct to the cell which contains a βc mutant having a mutation at the Tyr577 or an equivalent position.

Applicants have found that a single amino acid substitution in the common beta chain of the GM-CSF/IL-3/IL-5 receptors has a profound effect on haematopoietic cell behaviour that results in increased cell survival and proliferation. Primary foetal liver cells from mice devoid of endogenous beta common and beta IL-3 were used and transduced with an IRES construct containing both the alpha chain and the beta chain of the human GM-CSF receptor. Foetal liver cells transduced with a beta chain mutant Tyr577Phe exhibited increased survival both in the absence and in the presence of human GM-CSF. In addition they formed a much larger number of day 14 colonies which were larger in size. Delta assays showed an expansion of day 14 colony-forming cells after seven days in liquid culture with GM-CSF. Increased cell proliferation was also seen in CTL-EN cells transduced with the Tyr577Phe mutant receptor as demonstrated by a shift to the left in the response to human GM-CSF. Tyr577Phe mutant did not alter the recruitment of SHIP-2 to the receptor, the activation of JAK-2 or the phosphorylation of STAT-5, however, mutation of Tyr577 failed to recruit Shc to the receptor and to promote its tyrosine phosphorylation.

Significantly, a profound effect on the activation of raf and in the activation of the SH-2-containing inositol phosphatase SHIP was found.

Cells having an introduced mutation of the Tyr577 or an equivalent residue of the receptor, and preferably the βc of the receptor may be used in assays to screen for compounds that can affect cell survival.

In a further preferred embodiment, the present invention provides a method of modulating proliferation in a cell said method including
introducing a modification to a binding motif capable of binding to a cytoplasmic protein said motif consisting of the following amino acid sequence:
   N—X—X—Y
   wherein X is any residue, and Y is tyrosine.

In a further preferred embodiment of the present invention, there is provided a method of modulating proliferation in a cell, said method including
introducing a modification to a tyrosine of a binding motif capable of binding to a cytoplasmic protein said motif consisting of the following sequence:
   N—X—X—Y
   wherein X is any residue, and Y is tyrosine and wherein the tyrosine is equivalent to Tyr577 of the common beta chain (βc) of the GM-CSF receptor or an equivalent thereof.

Modification of the Tyr577 or an equivalent may be conducted by any means herein described. However, any means that uncouples an interaction of Tyr577 or an equivalent to the cytoplasmic protein Shc or SHIP-2 will be most preferred to cause modulation of proliferation. Full uncoupling of the Shc or SHIP-2 cytoplasmic protein may lead to complete inhibition of phosphorylation of the Tyr577 or an equivalent. The applicants have found that debilitating this event leads to an increased cellular activity and proliferation resulting in increased survival and larger colonies.

Accordingly, in yet another preferred embodiment, the invention provides a method of modulating activity in a cell, said method including
modifying phosphorylation of a tyrosine of a binding motif capable of binding to a cytoplasmic protein said motif consisting of the following amino acid sequence:
   N—X—X—Y
   wherein X is any residue, and Y is tyrosine.

In a further preferred embodiment of the present invention, there is provided a method of modulating activity in a cell, said method including
modifying phosphorylation of a tyrosine of a binding motif capable of binding to a cytoplasmic protein said motif consisting of the following amino acid sequence:
   N—X—X—Y
   wherein X is any residue, and Y is tyrosine and wherein the tyrosine is equivalent to Tyr577 of the common beta chain (βc) of the GM-CSF receptor or an equivalent thereof.

Preferably the cellular activity is cell growth and/or proliferation. Both activities can lead to larger cells and larger colonies.

The modulation of the phosphorylation events which phosphorylate the tyrosine on the binding motif will affect the binding of a cytoplasmic protein which in turn will affect the activation of signalling molecules which activate a cascade of events leading to cell signalling pathways and cellular activities. Preferably the cellular activities are selected from the group including mitogenesis, proliferation, transformation, differentiation, cell survival, chemotaxis, motility, enhanced phagocytosis, bacterial killing, superoxide production and cytotoxicity. Most preferably, the cellular activity is proliferation or cell survival. More preferably it is cell survival.

The modification of phosphorylation of the Tyr577 or an equivalent may be an increase or a decrease of the phosphorylation of the residue. Methods of increasing or decreasing (inhibiting) phosphorylation are known to those skilled in the art. However, specifically, the use of specific kinase inhibitors are preferred to inhibit the phosphorylation.

Most preferably Tyr577 of βc is modified by phosphorylation. By inhibiting phosphorylation of Tyr577 cellular proliferation can be increased. Conversely, by inducing phosphorylation, cell growth can be inhibited.

Although not wishing to be limited by theory, it is perceived that phosphorylation of Tyr577 of βc may improve the binding of a cytoplasmic protein such as Shc or SHIP-2 to the residue so that when the cytoplasmic protein is reacted with the residue or equivalent thereof, binding may occur to bring other cytoplasmic proteins or signalling molecules into close proximity to the receptor. Phosphorylation may occur by any means which transfers a phosphoryl (phosphate) group to the Tyr577.

In yet another preferred aspect of the present invention, there is provided a method of increasing cell growth, said method including
inhibiting activation of a tyrosine of a binding motif capable of binding to a cytoplasmic protein said motif consisting of the following amino acid sequence:
   N—X—X—Y
   wherein X is any residue, and Y is tyrosine.

In a further preferred embodiment of the present invention, there is provided a method of increasing cell growth, said method including
inhibiting activation of a tyrosine of a binding motif capable of binding to a cytoplasmic protein said motif consisting of the following amino acid sequence:
   N—X—X—Y
   wherein X is any residue, and Y is tyrosine and wherein the tyrosine is equivalent to Tyr577 of the common beta chain (βc) of the GM-CSF receptor or an equivalent thereof.

As found in the present invention the tyrosine of the motif and more preferably Tyr577 or an equivalent is pivotal in the activation of mechanisms that are essential to the survival of cells and most preferably of cell growth and proliferation. Increased cell growth leads to increased cell colonies and the methods described herein are also useful for increasing colony size of the cells.

The cells are most preferably haematopoietic cells as described above, and are cells that incorporate the GM-CSF receptor and more specifically, the common βc. However, other cells may also be modified using the motif as herein described.

It has been described above that loss of effect of Tyr577 or an equivalent either by substitution, deletion or insertion, inhibition or by non-activation of phosphorylation, can cause an increase in cell growth and colony size. These forms of inhibiting activation of Tyr577 or an equivalent are within the scope of the present invention.

It is preferred that antagonists that bind to Tyr577 or an equivalent in either the phosphorylated or unphosphorylated form can be used to inhibit activation of Tyr577 or an equivalent. By this it is meant that Tyr or Tyr577 or an equivalent can lose its ability to bind a cytoplasmic protein and further activate cell signalling pathways. This may be useful to increase cell survival or activation. Preferably antagonists may be useful to increase cell survival or activation by preventing phosphorylation preferably by preventing Tyr577 or an equivalent phosphorylation of β_{c} or equivalent thereby preventing the cytoplasmic protein binding to the binding motif. Alternatively, they may prevent the interaction of a signalling molecule binding to a phosphotyrosine bound Shc or equivalent.

Prevention of phosphorylation of β_{c} or Tyr577 or an equivalent as described above may also inhibit activation of Tyr577 or an equivalent and this may be achieved by inhibition of the specific kinases involved in the phosphorylation of the Tyr577 or an equivalent residue or it may include mutation of the residue to prevent the cytoplasmic protein such as Shc or SHIP-2 from binding and activating cell cycle pathways. Kinase inhibitors such as H89 which binds to PKA may be used. Preferably, cell permeable kinase inhibitors are used.

Antagonists may include antibodies, small peptides, small molecules, peptide mimetics or any type of molecule known to those skilled in the art that are directed to the Tyr577 or an equivalent so as to prevent attachment of cytoplasmic proteins such as Shc, SHIP-2 to a Tyr residue or a signalling molecule. Antibodies may be generated in response to any of the Tyr577 or an equivalent described above by methods known and available to the skilled addressee.

However, the invention may further encompass modifying the cytoplasmic proteins that also bind to Tyr577 or an equivalent. Without affecting Tyr577 or an equivalent in the cell, modifications can be directed at the cytoplasmic proteins or other factors which may cause phosphorylation of the Tyr577 or an equivalent. Effectively, the invention includes any means which can cause an uncoupling of the interaction between Tyr577 or an equivalent and its cytoplasmic proteins, namely Shc and SHIP-2.

In yet another aspect of the present invention there is provided a method of transplantation of cells, said method including
inhibiting activation in the cell of a tyrosine of a binding motif capable of binding to a cytoplasmic protein said motif consisting of the following amino acid sequence:
   N—X—X—Y
   wherein X is any residue, and Y is tyrosine; and
transplanting the cells into a patient in need of such treatment.

In a further preferred embodiment of the present invention, there is provided a method of transplantation of cells, said method including
inhibiting activation in the cell of a tyrosine of a binding motif capable of binding to a cytoplasmic protein said motif consisting of the following amino acid sequence:
   N—X—X—Y
   wherein X is any residue, and Y is tyrosine and wherein the tyrosine is equivalent to Tyr577 of the common beta chain (βc) of the GM-CSF receptor or an equivalent thereof; and
transplanting the cells into a patient in need of such treatment.

This method of inhibiting activation of Tyr577 or an equivalent to increase cell growth may be conducted in vivo or in vitro. Cell culture techniques to increase cell populations may be enhanced by the method described herein, by inhibiting the interaction of Tyr577 or an equivalent and the cytoplasmic protein Shc or SHIP-2. This would be particularly useful for expanding colonies of cells and progenitor cells in vitro prior to injection into a patient. However the interaction of Tyr577 or an equivalent may be directed in vivo by the use of specific kinase inhibitors.

The inhibition of the activation in the cell may be useful for enhancing transplantation efficiency. By increasing proliferation of the cell by inhibiting activation, more cells may successfully incorporate and integrate in the transplant.

Similarly, cells in the region of the transplantation may be treated to inhibit activation Tyr577 or an equivalent so as to increase cell proliferation and growth in that region to enhance cell growth and reduce rejection of the transplant. This may assist in the graft “taking” and possibly reduce the number of cells required to transplant. If a lesser number of cells is transplanted, the method is useful to expand colony forming cells which is useful for providing protection after bone marrow transplantation, particularly in the time between stem cell engraftment and bone marrow recovery.

Patients undergoing transplantation may be treated with inhibitors of phosphorylation and directed toward the area of transplantation so as to increase cell growth or enhance cell growth which may improve the chances of the grafted tissue for survival.

In another aspect of the present invention there is provided a method of improving wound healing in a patient, said method including
inhibiting activation of a tyrosine of a binding motif capable of binding to a cytoplasmic protein said motif consisting of the following amino acid sequence:
   N—X—X—Y
   wherein X is any residue, and Y is tyrosine in a region of the wound.

In a further preferred embodiment of the present invention, there is provided a method of transplantation of cells, said method including
inhibiting activation in the cell of a tyrosine of a binding motif capable of binding to a cytoplasmic protein said motif consisting of the following amino acid sequence:
   N—X—X—Y
   wherein X is any residue, and Y is tyrosine and wherein the tyrosine is equivalent to Tyr577 of the common beta chain (βc) of the GM-CSF receptor or an equivalent thereof in a region of the wound.

By inhibiting activation of Tyr577 or an equivalent of βc, the cells can be induced to proliferate in the region of the wound. As described above, there are methods for inhibiting the activation of Tyr577 or an equivalent and any of these methods described above may be used to inhibit Tyr577 or an equivalent and therefore uncouple Tyr577 from Shc or SHIP-2. Most preferred is an inhibition of phosphorylation of Tyr577 or an equivalent. Preferably, this may be achieved by using specific kinase inhibitors.

However, since phosphorylation is a process that is common to a number of cellular activities, the use of a general dephosphorylating agent may be harmful.

Accordingly, it is also preferred to utilise antagonists of Tyr577 or an equivalent which may inhibit the binding of Tyr577 or an equivalent to the cytoplasmic protein Shc or SHIP-2. The use of these molecules can target and specifically direct their inhibition of the phosphorylation and subsequent events which can assist in the increased cell growth and proliferation for wound healing.

In yet another aspect of the present invention, there is provided a method for screening cell growth promoting compounds, said method including
providing a cell having a receptor containing a βc having a Tyr577 residue or equivalent;
inducing phosphorylation of the Tyr577 or an equivalent;
exposing the cell to the compound; and
assessing colony formation of the cells.

This aspect of the present invention utilises the finding that Tyr577 or an equivalent is central to the induction of cell growth and survival leading to larger colonies and that inhibition of phosphorylation is necessary to increase cell survival and produce larger colonies. Where the compound increases colony formation and reverses the effects of the induced phosphorylation, then it may be deduced that the compound has growth promotion properties.

Preferably the cell is a haematopoietic cell as herein described.

The present invention may also be used as a model for proliferative diseases. Given the interaction between the binding motif and the cytoplasmic proteins, any part of the interactions can be monitored to determine any aberration between the cells in question and that of a normal cell. Aspects of the model may analyse the phosphorylation ability of the Tyrosine residues or analyse the interaction between the respective cytoplasmic proteins of Shc and SHIP-2 both with Tyrosine.

Preferably the model is based on a haematopoetic cell as described above. However, any other cell that contains the motif may be used. More preferably, the cell has a GM-CSF receptor including the Tyr577 or an equivalent upon which the model is based.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

Examples of the procedures used in the present invention will now be more fully described. It should be understood, however, that the following description is illustrative only and should not be taken in any way as a restriction on the generality of the invention described above.

### EXAMPLES

### Example 1

### Y577F Mutation Leads to Enhanced Numbers of Colonies in Response to GM-CSF

(a) Cell Lines and Primary Cells

The murine factor dependent cell line CTL which has been described previously {Le, Stomski, et al. 2000 4135/id} was used for the proliferation studies. These cells were maintained in RPMI-1640 with 10% foetal calf serum (FCS) and murine IL-2 generated from an E-coli expression system.

Psi-2 ecotropic retrovirus packaging cells {Mann, Mulligan, et al. 1983 3888/id} were maintained in DMEM supplemented with 10% FCS. Transfected pools of cells were selected and maintained in the above medium plus geneticin sulphate (G418) at 400 μg/ml. were transfected with the RufNeo retroviral vector {Rayner & Gonda 1994 4136/id} containing wild type or mutant hoc.

Murine foetal liver cells were harvested at dpc 13.5 from mice that were knockout for both the common beta chain (β_{c}) and the IL-3-specific beta chain (β_{IL-3}) {Scott, Robb, et al. 2000 2960/id}. These cells were cultured in IMDM supplemented with 15% FCS.

(b) Generation of Murine Cells Expressing Human GM-CSF Receptor

CTL-EN or murine foetal liver cells were engineered to express both alpha and beta chains of the GM-CSF receptor using a retroviral transduction system.

For generation of the retroviral particles, the backbone plasmid was based on pRUFneo and the GM-CSF receptor subunits were cloned into the multiple cloning site. Additionally an IRES was inserted to allow expression of both receptor subunits in a single infection event. Producer cell lines were generated in psi-2 cells by calcium phosphate transfection followed by FACS to generate cell lines with high expression of both receptor subunits.

Target cells (CTL or foetal livers) were co-cultivated with the irradiated psi-2 cells for 48 hours and then harvested and cultured for an additional 24 hours with fresh medium added. Transduction levels were assessed using immunofluorescence analysis with biotinylated antibodies specific to the GM-CSF receptor alpha chain and streptavidin-PE. 5000-10000 cells were analysed using a Coulter Epics XL.

(c) Colony Assays

The response of immature cells to GM-CSF was analysed using colony formation assays. Foetal liver cells were transduced and expression of the GM-CSF receptor quantitated as described above. Colony forming cells were assayed using a double layer agar assay. Plates were prepared with underlayers comprised of IMDM supplemented 0.5% agar (Difco) containing cytokines as shown in the figures. An overlayer was added with transduced cells, at a concentration of 100,000 per dish, in 0.3% agar. All media contained 10% heat inactivated FCS (JRH Biosciences) with penicillin and streptomycin added.

All cytokines were diluted in PBS and a control of PBS alone was added in each assay to verify that colonies were formed in response to cytokine stimulation. Additionally, all assays included a general stimulus composed of a cocktail of IL-6 (100 ng/ml), SCF (100 ng/ml) and erythropoietin (4 U/ml).

Plates were incubated at 37° C. 5% CO₂ for 14 days and colonies counted using an inverted microscope at the completion of this time.

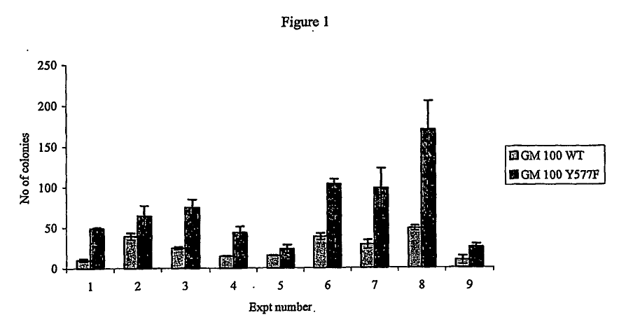

FIG. 1 shows colony formation from foetal livers transduced with GM-CSF receptors. 100,000 cells were plated in 35 mm dishes with GM-CSF at 100 ng/ml. Colony numbers were enumerated after incubation at 37° C. 5% CO₂ for 14 days. The figure shows data from 9 separate experiments with triplicate plates in each. Average and standard deviations are represented in the figure.

### Example 2

### Y577F Point Mutation can Give Rise to Larger Colonies than Wild Type GM-CSF Receptor Beta Chain

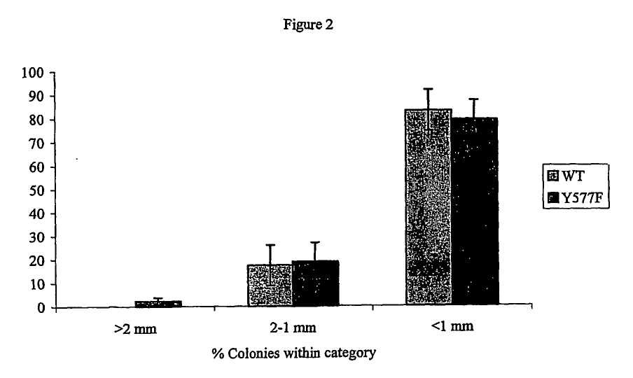

FIG. 2 shows colonies from 2 of the assays shown in Example 1 were analysed according to their overall size. Stained dishes were categorised into 3 groups; 1), colonies greater than 2 mm 2) between 1 and 2 mm and 3) those smaller than 1 mm. Data is represented for each group as the percent of the total number of colonies and FIG. 2 shows mean value with standard deviation.

### Example 3

### Foetal Liver Cells Transduced with Y577F Mutant of the Beta Chain Form Greater Numbers of Colonies at all Concentrations of GM-CSF

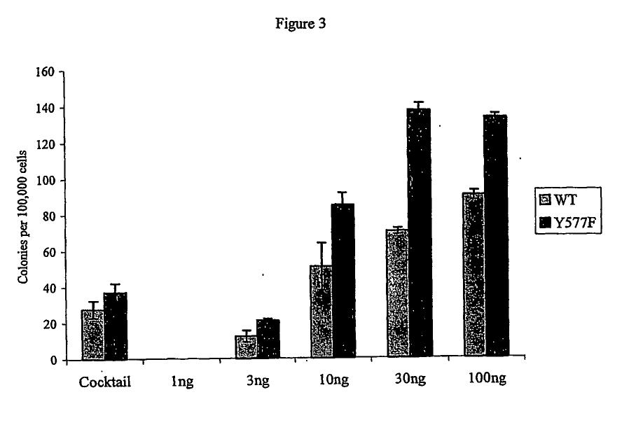

A titration of GM-CSF was used to assess the response of the cells to lower concentrations of cytokine in addition to the higher concentrations reported above. FIG. 3 shows that there are greater numbers of colonies.

### Example 4

### Delta Assay of Cells Cultured for 7 or 14 Days Prior to Colony Formation

(a) Delta Assay

An extended colony assay was performed in which cells were either plated as described above on Day 0 or cultured in GM-CSF or a cocktail of IL-6 (100 ng/ml), SCF (100 ng/ml) and G-CSF (10 ng/ml) for 7 or 14 days. On Days 7 or 14 cells were plated in colony assays exactly as previously. All colonies were enumerated after 14 days incubation at 37° C. 5% CO₂.

(b) 7 Days Prior to Colony Formation

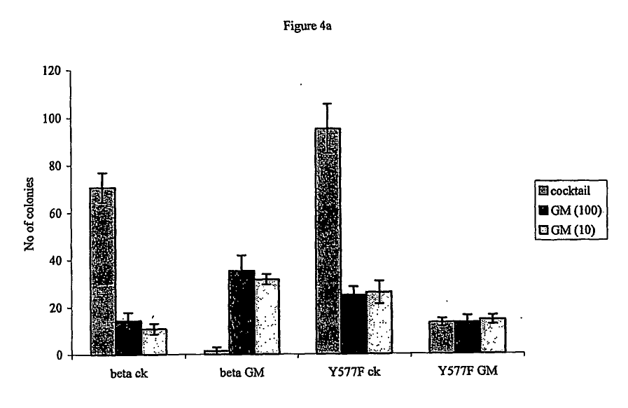

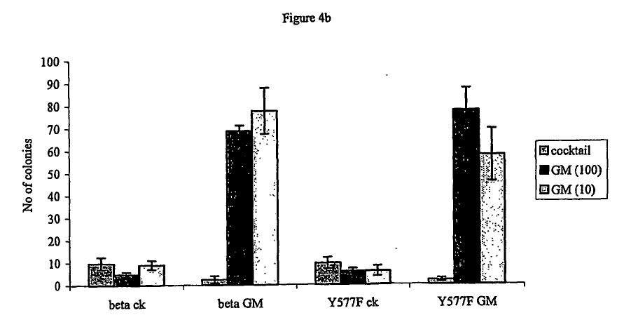

Cells were cultured in liquid media supplemented with either GM-CSF or cocktail for 7 days prior to plating in agar. As can be seen from the groups grown in GM-CSF and in FIG. 4a, as denoted by GM on the x-axis, there is greater response to cocktail stimulation for the Y577F group compared to the wild type receptor. Response is similar for cells grown in cocktail for the 7 days prior to plating.

(c) 14 Days Prior to Colony Formation

Cells were cultured in liquid media supplemented with either GM-CSF or cocktail for 14 days prior to plating in agar. As can be seen from the groups grown in GM-CSF and in FIG. 4b, as denoted by GM on the x-axis, there is greater response to cocktail stimulation for the Y577F group compared to the wild type receptor. Response to cocktail but not GM-CSF is also greater for this group for cells grown in cocktail for the 14 days prior to plating.

### Example 5

### CTL Cells Expressing the Human GM-CSF Receptor Have Greater Response to GM-CSF when Y577 is Mutated to Phenylalanine

(a) Proliferation Assay

The proliferative response of CTL cells expressing wild type or mutant GM-CSF receptor beta chains was measured in microwell assays of 1000 cells stimulated with serially diluted quantities of GM-CSF. CTL cells are grown in IL-2 but were starved of growth factor for 24 hours before setting up the proliferation assays as described previously {Sun, Woodcock, et al. 1996 3188/id}. The ³H-Thymidine incorporation was determined by liquid scintillation.

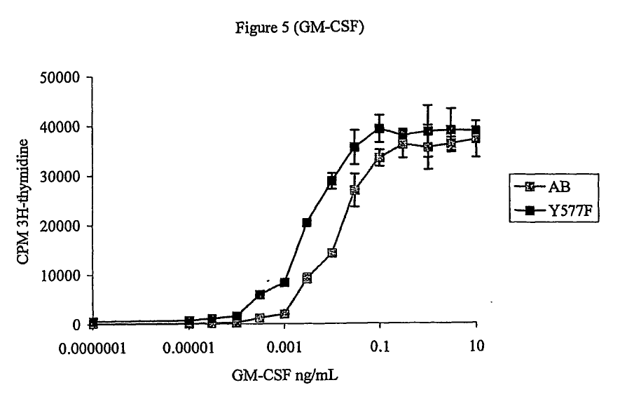

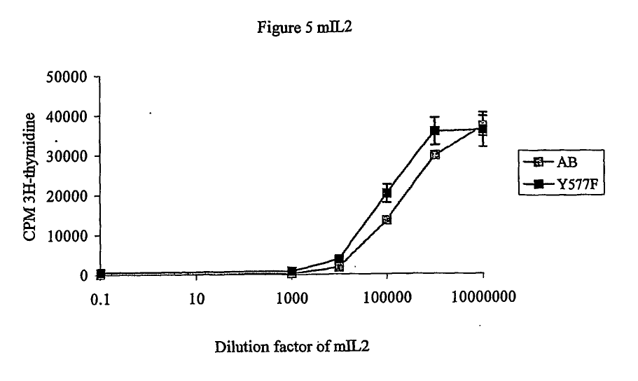

FIG. 5 shows a proliferation assay for CTL cells transduced with the human GM-CSF receptor. The cells were starved of cytokine for 24 hours and then stimulated with a titration of human GM-CSF or murine IL-2 for 48 hours. Proliferation was quantitated using ³H-Thymidine incorporation and liquid scintillation counting. The reduction in ED50 for can clearly be seen for Y577F when compared to wild type beta chain for response to GM-CSF but not for IL-2.

Finally it is to be understood that various other modifications and/or alterations may be made without departing from the spirit of the present invention as outlined herein.

1. A binding motif capable of binding to a cytoplasmic protein said motif comprising the following amino acid sequence:
N—X—X—Y
wherein X is any residue, and Y is a tyrosine residue or an equivalent thereof.

2-7. (canceled)

8. The binding motif of claim 1 wherein the sequence comprises the common beta chain (βc).

9. The binding motif of claim 1 wherein the tyrosine residue is equivalent to residue Tyr577 of the common beta chain (βc).

10. The binding motif of claim 1 having a modification at a residue equivalent to the tyrosine residue.

11. The binding motif of claim 1 wherein the residue equivalent to the tyrosine residue is substituted with a phenylalanine residue.

12-13. (canceled)

14. A method of modulating cellular activity in a cell, said method comprising:
introducing a modification to a binding motif capable of binding to a cytoplasmic protein said motif comprising the following amino acid sequence:
N—X—X—Y
wherein X is any residue, and Y is a tyrosine residue.

15. (canceled)

16. The method of claim 14 wherein the tyrosine residue is equivalent to Tyr577 of the common beta chain (βc).

17. The method of claim 16 wherein the common beta chain (βc) is of the GM-CSF/IL-3/IL-5 receptor.

18. The method of claim 14 wherein the activity is modulated by introducing a modification of phosphorylation of the tyrosine residue of the motif.

19. The method of claim 18 wherein the phosphorylation is increased by subjecting the cell to a phosphorylating agent.

20. (canceled)

21. The method of claim 18 wherein the phosphorylation is decreased by mutating, substituting, or deleting the tyrosine residue.

22. The method of claim 21 wherein the tyrosine residue is substituted for phenylalanine.

23. The method of claim 18 wherein the phosphorylation is decreased by subjecting the cell to an antagonist which inhibits phosphorylation of the tyrosine residue.

24. The method of claim 18 wherein the phosphorylation is decreased by subjecting the cell to a kinase inhibitor to inhibit phosphorylation of the tyrosine residue.

25. (canceled)

26. The method of claim 14 for modulating cellular activity, said method further comprising inhibiting binding of a cytoplasmic protein to the motif.

27-28. (canceled)

29. The method of claim 18 for modulating cellular activity, said method comprising activating cellular activity by inducing phosphorylation of the tyrosine residue of the motif.

30. The method of claim 14 wherein the cellular activity is selected from the group comprising: cell survival; proliferation; differentiation; mitogenesis; transformation; chemotaxis; motility; enhanced phagocytosis; enhanced bacterial killing; superoxide production; and cytoxicity.

31-57. (canceled)

58. A method for screening of cell growth promoting compounds, said method comprising:
obtaining a cell having a receptor containing the common beta chain (βc) having a Tyr577 residue or equivalent;
inducing phosphorylation of a tyrosine residue in a binding motif capable of binding to a cytoplasmic protein said motif consisting of the following amino acid sequence:
N—X—X—Y
wherein X is any residue, and Y is a tyrosine residue or an equivalent thereof;
exposing the cell to the compound; and
assessing colony formation of the cells.

59. The method of claim 58 wherein the tyrosine residue is equivalent to Tyr577 of the common beta chain (βc).

60. The method of claim 58 wherein the common beta chain (βc) is of the GM-CSF/IL-3/IL-5 receptor.
